SEQUENCE LISTING (2 sequences)

SEQ ID NO: 1 - DNA, 20 bp, Artificial Sequence
  primer_bind: synthetic oligonucleotide primer complementary to viral sequence
cgagtcaacacgggtgaaga

SEQ ID NO: 2 - DNA, 20 bp, Artificial Sequence
  primer_bind: synthetic oligonucleotide primer complementary to viral sequence
cgtgtcagtcatgtgtcgct

(19) United States
(12) Patent Application Publication
(10) Pub. No.: US 2004/0001863 A1
(43) Pub. Date: Jan. 1, 2004

(54) Immortal cell line derived from grouper Epinephelus coioides and its applications therein

(76) Inventor: Shau-Chi Chi, Taipei (TW)

Correspondence Address: VENABLE, BAETJER, HOWARD AND CIVILETTI, LLP, P.O. BOX 34385, WASHINGTON, DC, 20043-9998, US

(21) Appl. No.: 10/004,432

(22) Filed: Dec. 6, 2001

Related U.S. Application Data
(63) Continuation-in-part of application No. 09/450,696, filed on Nov. 30, 1999, now Pat. No. 6,436,702.
(60) Provisional application No. 60/110,699, filed on Dec. 3, 1998.

(51) Int. Cl. A61K 39/12

(52) U.S. Cl. 424/204.1

(57) ABSTRACT

The present invention provides for fish vaccines and methods of using the fish vaccines to immunize susceptible fish against viral infections. In particular, the present invention is directed to vaccines and methods of immunizing susceptible fish for two aqua-viral diseases, the viral nervous necrosis (VNN) disease which is caused by nervous necrosis virus (NNV), and infectious pancreatic necrosis (IPN) disease which is caused by infectious pancreatic necrosis virus (IPNV). The vaccines are produced by inactivated viruses which in turn were produced from treatment of viruses that are mass-produced in an immortal cell line derived from Epinephelus coioides having an ATCC deposit number of PTA-859.

### RELATED APPLICATION

This application is a continuation-in-part application which claims the priority of U.S. Provisional Patent Application No. 60/110,699, filed on Dec. 3, 1998, and U.S. patent application Ser. No. 09/450,696, filed on Nov. 30, 1999, which are incorporated herein by reference.

### FIELD OF THE INVENTION

The present invention relates to vaccines for immunizing susceptible fish against infection by Infectious Pancreatic Necrosis Virus (IPNV) and Nervous Necrosis Virus (NNV). The present invention also relates to methods of preparing the vaccine for immunizing fish against IPNV and NNV. Both the IPNV and NNV are mass-produced in an immortal cell line (GF-1) derived from the fin tissue of grouper Epinephelus coioides. The IPNV or NNV vaccines are produced from inactivated viruses. The present invention also relates to methods for immunizing susceptible fish against IPNV or NNV infection.

### BACKGROUND OF THE INVENTION

Nervous necrosis virus (NNV), a pathogen found in many varieties of hatchery-reared marine fish, has caused mass mortality of such fish at their larval or juvenile stages. NNV belongs to the family Nodaviridae. Fish nodaviruses isolated from different species (such as SJNNV, BFNNV, JFNNV, TPNNV, RGNNV, GNNV etc.) are closely related to each other owing to the high similarity of the conserved region of their coat protein genes. NNV, also named as fish encephalitis virus (FEV) and piscine neuropathy nodavirus (PNN), is an unenveloped spherical virus with particles sized between 25 and 34 nm. The virus is characterized by vacuolation of the nerve tissues. Viral Nervous Necrosis (VNN) disease has been found in many countries under various names such as viral fish encephalitis, fish encephalomyelitis, cardiac myopathy syndrome. The hosts of NNV include many species of marine fish, for example: parrotfish, sea bass, turbot, grouper, stripped jack, tiger puffer, berfin flounder, halibut, barramundi, and spotted wolffish.

According to the statistics shown in 1993, approximately 159 fish cell lines have been established which have demonstrated a capacity for growing fish viruses (Fryer and Lannan, J. Tissue Culture Method (1994), 10:57-94). Most of these cell lines are derived from the tissues of freshwater fish. There are only thirty-four cell lines that are originated from marine fish. Although some of the fish cell lines, which include RTG-2, CHSE-214, BF2, SBL, FHM, EPC, have been tested for the susceptibility of fish nodavirus, none of these cells lines has shown cytopathic effects (CPE) after viral inoculations.

In 1996, SSN-1 cell line, a cell line derived from striped snakehead Channa Striatus, has been successfully used for isolating sea bass nodavirus (Frerichs et al., J. General Virology (1996)77:20672071). However, SSN-1 cell line has been known to be persistently contaminated with C-type retrovirus (Frerichs et al., J. General Virology (1991) 72:2537-2539). Therefore, it is not suitable for the production of fish nodavirus.

Infectious pancreatic necrosis virus (IPNV) is the prototype virus of the family Bimaviridae. Bimaviruses also include infectious bursal disease virus of domestic fowl and drosophila X virus of Drosophila melanogaster. The virus is capable of infecting a number of different hosts and has a worldwide presence. For example, IPNV has been found in a variety of fish species throughout the world, including various trout and salmon species, carp, perch, pike, eels and char, as well as mollusks and crustaceans, although so far acute diseases has only been reported in a limited number of salmonid species, such as trout and salmon. Pilcher et al., Crit. Rev. Microbiol. (1980) 7:287-364.

An outbreak of IPNV in a hatchery can be an economic disaster for the aquaculturist. IPNV attacks young fish (usually two- to four-month old), resulting in high mortality. Frantsi et al., J. Wildlife Dis. (1971)&:249-255. In trout, IPNV usually attacks young fry about five to six weeks after their first feeding. The affected fish are darker than usual, have slightly bulging eyes and often have swollen bellies. At the beginning of an outbreak, large numbers of slow, dark fry are seen up against water outflows, and fish are seen “shivering” near the surface. The pancreas appears to be the primary target organ for the virus, with the pancreatic fat cells or Islets of Langerhans being unaffected. The intestine is the only organ besides the pancreas where viral lesions are consistently found. McKnight et al., Br. Vet. J. (1976)132:76-86.

After an IPNV outbreak, the surviving fish generally become carriers of the virus. The persistence of the virus in carrier fish appears to be due to continuous viral production by a small number of infected cells in certain organs. The surviving carrier fish become even more economically disastrous to aquaculturists because the only control method currently available for eliminating the virus in carrier fish is to completely destroy these fish.

Viral diseases cannot be cured by therapeutic reagents. The best ways to contain viral diseases include prevention through early detection and the development of vaccines. In either way, the understanding of the biological, biochemical, and serological characteristics of the virus is fundamentally required, which in turn relies on the industry to have the capacity of mass producing the pure form of viruses, preferably through an in vitro cell culture system. Therefore, the development of a new cell line which can be susceptible to fish viruses, particularly NNV and IPNV, is desperately in demand in order to control the wide spread of fish viral diseases due to viral infection.

Grouper is an important hatchery fish in Taiwan. In recent years, there have been several reports regarding the establishment of cell lines derived from grouper. For example, Chen et. al. (Japan Scientific Society Press (Tokyo) (1988) 218-227) have reported their establishment of several cell lines from the fin and kidney tissues of grouper Epinephelus awoara. Lee (Master Thesis from the Department of Zoology at the National Taiwan University, 1993) also has reported his establishment of the cell lines derived from the eye pigment cells and brain tissue of grouper Epinephelus amblycephalus. However, Chen et al. do not provide sufficient data in support of the claim for immortality in their cell lines and Lee expressly indicates in his thesis that his grouper cell lines are not immortal. Moreover, neither Chen et al.'s nor Lee's cell lines are susceptible to fish nodavirus.

Recently, severe mortality among groupers has repeatedly occurred which is caused primarily by nodavirus. As present, fish nodavirus has been discovered in grouper and can be isolated from moribund grouper which possess symptoms of VNN disease (Chi et al., J. Fish Disease (1997) 20:185-193). Electron microscopic examination of the tissues from grouper shows that, in addition to nodavirus infection, grouper is susceptible to other viral infections (Chi, COA Fisheries Series No.61, Reports on Fish Disease Research (1997) 18:59-69).

IPNV is not a virus commonly found in groupers. IPNV was originally reported from Canada as catarrhal enteritis in brook trout (Salvelinus fontinalis) and later from USA as an acute viral disease in hatchery-reared brook trout fry at start feeding. Since then, the disease has spread to most salmonid farming countries. The first report of IPNV-related pathology in Atlantic salmon came from Scotland. The same year clinical IPNV with high mortality in post-smolt and IPNV with 30% mortality in an Atlantic salmon hatchery were reported from Norway. In Norway, the incidence of clinical infectious pancreatic necrosis (IPN) disease in Atlantic salmon farms was 39% in 1991, and it had increased to 61% in 1995. Christie, Fish Vaccinology (1997) 90:191-199.

At this time, there are a few cell lines that are capable of producing IPNV. The most commonly known one is Chinook salmon embryo (CHSE) cell line, which is available commercially and can be obtained from the American Type Culture Collection (USA). Recently, there has been reported that another cell line derived from Penaeus Monodon is also susceptible to IPNV. See U.S. Pat. No. 6,143,547 to Hsu. However, although these cell lines have demonstrated susceptibility to IPNV, the viral titers produced by these cell lines are allegedly low.

In the invention to be presented below, an immortal cell line (GF-1 cell line) derived from the fin tissue of grouper Epinephelus coioides (Hamilton) will be introduced: The GF-1 cell line of the present invention is susceptible to various viruses, particularly fish nodavirus such as NNV. In addition to the viruses that are commonly found in groupers, the GF-1 cell line is capable of replicating IPNV in high titers, even though IPNV is not a virus that commonly known to cause diseases in groupers. Therefore, both NNV and IPNV can be mass-produced using the GF-1 cell line, which are suitable for use in antibody and vaccine production to protect fish from viral infections.

### SUMMARY OF THE INVENTION

The present invention provides vaccines for immunizing susceptible fish against viral infections. In particular, the vaccines are against nervous necrosis virus (NNV) or infectious pancreatic necrosis virus (IPNV). The vaccine contains an immunogenically effective amount of a non-infective virus that is modified to become non-infective. The virus is produced in an immortal cell line (GF-1) from Epinephelus coioides having an ATCC deposit No. PTA-859.

The non-infective vaccines are prepared by inactivated virus. The preferred vaccination route is by oral administration or intraperitoneal or intramuscular injection.

NNV is susceptible to a lot of marine fish, which include, but are not limited to, parrotfish, sea bass, turbot, grouper, stripped jack, tiger puffer, berfin flounder, halibut, barramundi, and spotted wolffish. The most susceptible fish for NNV is grouper.

IPNV is susceptible to salmonids, particularly trout, salmon, carp, perch, pike, and eel. The most susceptible fish for IPNV are trout and salmon, such as rainbow trout (Oncorhynchus mykiss), brook trout (Salvelinus fontinolis), chinook salmon (Oncorhynchus tshawytscha), coho salmon (Oncorhynchus kisutch), sockeye salmon (Oncorhynchus nerca) and Atlantic salmon (Salmo salar).

The preferred method for preparing inactivated NNV is by heat treatment. A 60-100°C. treatment for at least 1 hour should be sufficient to kill or inactivate NNV. The preferred method for preparing inactivated IPNV is by formalin treatment. An incubation of IPNV for 7 days at 22° C. in a 0.5% formalin should be sufficient to kill or inactivate IPNV.

The present invention also provides a method for immunizing susceptible fish against viral infection by administering an immunogenic effective amount of vaccine to the susceptible fish. The vaccine contains a virus that is in the immortal cell line (GF-1) from Epinephelus coioides having an ATCC deposit No. PTA-859.

### BRIEF DESCRIPTION OF THE DRAWINGS

FIG. 1 shows the morphology of the GF-I cells observed under an inverted microscope. (A) A semi-confluent monolayer where both fibroblast-like and epithelial cells co-existed, and (B) a confluent monolayer of GF-1 cells at subculture 80 where fibroblast-like cells were the predominant cells. In the figure, an arrowhead indicates fibroblast-like cells. Similarly, an arrow indicates epitheloid cells. Bar=10 μm

FIG. 2 shows the chromosome number distribution of the GF-1 cells at (A) subculture 50, and (B) subculture 80.

FIG. 3 shows the effect of fetal bovine serum (FBS) on the growth rate of GF-1 cells at (A) subculture 50, and (B) subculture 80.

FIG. 4 shows the effect of temperature on the growth rate of GF-1 cells at subculture 80.

FIG. 5 shows the cytopathic effects (CPE) of GF-1 cells at subculture 80 after infection by (A) IPNV AB strain, (B) IPNV SP strain, (C) IPNV VR299 strain, (D) IPNV EVE strain, (E) HCRV, (F) fish nodavirus GNNV isolate, and (G) HEVF, as compared with (H) Uninfected GF-1 cells.

FIG. 6 shows the agarose gel electrophoresis of the product by RT-PCR amplification using a pair of primers (SEQ ID NO: 1 and SEQ ID NO:2) specific to the target region T4 of fish nodavirus SJNNV. Lane 1, PCR product from GNNV-infected GF-1 cells; lane 2, PCR product from non-infected GF-1 cells. M: pGEM marker.

FIG. 7 is an electron micrograph of GNNV-infected GF-1 cells. Inclusion bodies (indicated by arrowhead) and numerous non-enveloped viral particles are shown in the cytoplasm. I: an inclusion body filled with viral particles. M: mitochondria, N: nucleus. Arrowhead indicates the viral particle. Bar=1 μm.

### DETAILED DESCRIPTION OF THE INVENTION

The present invention provides vaccines and methods for effectively protecting fish susceptible to viral diseases, in particular, viral nervous necrosis (VNN) disease and infectious pancreatic necrosis (IPN) disease. Susceptible fish are those species of fish of which a particular virus is a pathogen and in which the vaccines of the present invention are capable of inducing protective immunity, i.e., the fish is capable of being protected from such disease by vaccination with the vaccines.

VNN disease is caused by nervous necrosis virus (NNV), a kind of fish nodavirus. It has been found in many marine fish, such as parrotfish, sea bass, turbot, grouper, stripped jack, tiger puffer, berfin flounder, halibut, barramundi, and spotted wolffish.

IPN disease caused by infectious pancreatic necrosis virus (IPNV). It is a lethal disease in both hatchery-reared juvenile salmonids and nonsalmonid fish. Salmonid fish include, but are limited to, pacific salmon in general (Oncorhynchus sp.), such as rainbow trout (Oncorhynchus mykiss), brook trout (Salvelinus fontinolis), chinook salmon (Oncorhynchus tshawytscha), coho salmon (Oncorhynchus kisutch), sockeye salmon (Oncorhynchus nerca) and Atlantic salmon (Salmo salar).

The general practice in dealing with disease outbreak in fish is by destroying the infected stocks and decontaminating the hatchery facilities because there is no effective methods for controlling viral diseases in fish. Recently, there has been a great deal of interest in developing vaccines for treating fish. However, vaccine development requires a constant supply of large quantity of virus. Using live fish as hosts for virus is too costly. Also, there have been a genuine environmental concern of disease outbreak in fish if the virus-raising fish are not properly contained. Thus, using cell line to produce the virus is a much better alternative for vaccine production. However, most of the currently available cell lines do not have the capability of mass production of the virus, particularly for NNV and IPNV. Therefore, until recently, little progress has been made toward producing truly practical vaccines for VNN and IPN.

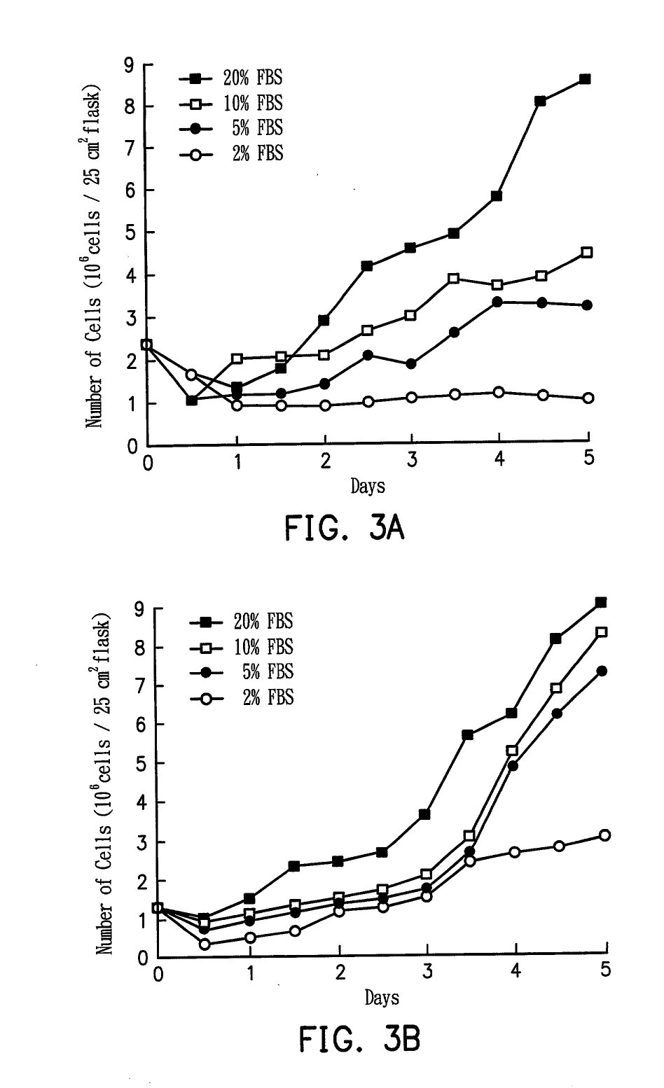

The GF-1 cell line of the present invention presents a great opportunity for vaccine development against NNV and/or IPNV. Although GF-1 is an immortal cell line derived from the fin tissue of grouper Epinephelus coioides (the fin tissue is not the target tissue for NNV in grouper; and grouper is not a susceptible fish for IPNV), the GF-1 cell line can produce both NNV and IPNV in great quantity. In fact, the titers of IPNV in GF-1 are about 100 times higher than those in CHSE-214 cell line, a well-known cell line derived from chinook salmon embryo which is by far the best known cell line for production of IPNV. In addition, GF-1 cell line is capable of forming confluent monlayer in as few as 3 days (see Example 1, infra), which is far much faster thant most of the other cell lines including CHSE-214. Furthermore, GF-1 cell line can be grown in a cell medium containing as low as 2% of fetal bovine serum (FBS)(See e.g., FIG. 3, infra), which makes the maintenance of the GF-1 cell line much more economically than other cell lines (most of the cell lines are maintained at about 10-20% FBS). Thus, using GF-1 cell line to mass produce NNV or IPNV opens the door for production of vaccines, particularly for NNV and IPNV.

There are generally two ways to prepare vaccines against viral infections. The first one involves the use of inactivated virus. The second one involves the use of attenuated virus.

Inactivated vaccines generally refer to a vaccine containing nonreplicating microorganisms or viruses that are noninfectious but which retain their protective antigens. Viral vaccines are usually inactivated by agents such as formalin, phenol, or β-propiolactone, or by temperature or extreme pH. When the virus becomes “inactivated,” it is sometimes referred to as “killed.”

The methods for preparing inactivated virus to be used in vaccine development are well known and a vaccine prepared by inactivated virus is normally effective in aquiring protective immunity. Protective immunity is referred to the condition induced by the administration of a vaccine to a fish so that the susceptibility of the fish to infection by a particular pathogen is reduced.

However, there is a fundamental problem in developing vaccines using inactivated virus, i.e., the lack of constant and sufficient supply of virus. In addition, as a prerequisite for preparing inactivated virus vaccine, the virus must be extensively purified in order to prevent cellular contamination. Thus, the preparing of a vaccine using inactivated virus can be costly. For fish hatcheries that operate on relatively thin profit margins, the additional expense for such vaccine can be detrimental to the industry.

The alternative to inactivated vaccine is probably the use of live attenuated virus. In general, the live attenuated vaccine is referred to a vaccine prepared from live microorganisms or viruses cultured under adverse conditions leading to loss of their virulence but retention of their ability to induce protective immunity. The traditional way of preparing attenuated virus for vaccine is to find a strain of virus that shows low virulence and would not cause outbreak of disease to be used as vaccine. For example, U.S. Pat. No. 4,053,582 to Stickl suggests the attenuation of fowl pox virus to form a new virus no longer pathogenic to fowl. This new virus is suggested to be capable of administration to animals and humans to treat a wide variety of infectious diseases.

Another method of attenuation is based on the sequential propagation of the virulent virus in a different cell line so as to produce an attenuated form of virus. For example, U.S. Pat. No. 4,783,411 to Gabliks discloses a method for preparing attenuated virus vaccine against influenza-A by passaging influenza-A virus in goldfish cell culture to reduce the infectivity of the virus and alter the antigenic characteristics of the virus. The attenuated virus is then used as a vaccine to innoculate mammalian species, thus conferring immunity.

The use of live attenuated virus imitates the natural viral infection. Thus, only small amounts of the antigen (i.e., the live attenuated virus) is needed because the virus can multiply in fish and the administration can be achieved by immersion.

The problem associated with using a live vaccine is that it is difficult to find a stable non-pathogenic strain of the virus. In fact, a strain of virus which is non-pathogenic in a target species can be pathogenic in other species if the virus is spread, which makes the use of live attenuated virus unsafe, especially if the live attenuated virus is administered to fish by immersion method.

For example, there have been several attempts to use live attenuated viral strains of IPNV for vaccine. See e.g., Dorson, International Conf. on IPNV, Taloires, France (1982). However, most of these reports suggested that the earlier attenuated strains either fail to infect the fish or fail to induce protective immunization. Strains with low virulence have also been tested as vaccines for more virulent strains, but mortality from the vaccinating strain was either too high or protection was only moderate. Hill et al., Fish Diseases, Third COPRAQ Session (W. Ahne, ed.), N.Y., pp. 29-36 (1980).

Recently, due to the well documented studies on the molecular structure of IPNV, See e g., Dobos, Annu. Rev. Fish Dis. (1995):5:25-54; Christie, Fish Vaccinology (1997) 90:191-199, new approach of generating live, nonpathogenic IPNV using recombinant RNA method has been reported. For example, U.S. Pat. No. 6,274,147 to Vakharia et al. discloses the development of a reverse genetic system for IPNV using plus-stranded RNA transcripts derived from cloned cDNA. Full-length cDNA clones of IPNV genome is constructed that contains the entire coding and non-coding regions of RNA segments A and B. Plus-sense RNA transcripts of both segments are prepared by in vitro transcription of linearized plasmids with T7 RNA polymerase. Transfection of chinook salmon embryo (CHSE) cells with combined transcripts of segments A and B generated live IPNV after 10 days of post-transfection.

IPNV contains a bisegmented double stranded RNA genome. The larger genome segment A (approximately 3100 bp) of IPNV has two open reading frames: (1) one large 2916 bp ORF encoding a 106 kD polypeptide which can be cleaved into at least three protein; and (2) one overlapping ORF of 444 bp encoding a 17 kD arginine rich polypeptide.

The three proteins encoded by the large ORF include: (1) a 60K to 62K precursor (pVP2) of the 52K to 54K major capsid protein VP2; (2) a 29K non-structural protein (NS); and (3) a 31K minor capsid protein VP3. VP3 is believed to be located internally, associated with the RNA, but may be partly exposed on the surface of the capsid. The localization of the 17 kD polypeptide is not known.

The smaller B segment (approximately 2900 bp) encodes a singe gene product (VP1) with a molecular weight of approximately 94K, presumed to be the viral RNA polymerase. VP1 is present as free polypeptide in the virion and as genome-linked protein, VPg.

Vakharia et al. discover that an NS-protein deficient virus can replicate, but will not induce lesions in host cells, and thus conclude that NS protein is directly involved in viral pathogenesis. The NS-protein deficient virus, thus, can be used as live attenuated vaccines for IPNV, which are nonpathogenic.

In addition, U.S. Pat. No. 5,165,925 to Leong discovers that fish can be immunized simultaneously in large numbers against IPNV infections by administering a vaccine containing IPNV VP2 polypeptide or an immunogenic portion of VP2. Also, a vaccine containing both VP2 (the major capsid protein) and VP3 (the minor capsid protein) is particularly effective. Such vaccines can be mass-produced by the culturing of bacterial host cells that contain expression vectors including IPNV cDNA sequences coding for the viral polypeptides in the vaccine.

Nevertheless, the neutralization ability of the attenuated vaccine produced by recombinant subunit vaccine is much worse than that of the inactivated vaccine (according to statistiscs, the vaccines produced from inactivated vaccine demonstrate about twice neutralization ability than that of the recombinant subunit vaccine). Also, recombinant subunit vaccines and DNA vaccines are not permitted to be sold in many western European countries.

Because the GF-1 cell line has the ability to mass produce the viruses, particularly NNV and IPNV, in an economical way, it is preferred to use the GF-1 cell line as a tool to mass produce the viruses for the preparation of inactivated vaccine.

The following examples are illustrative, and should not be viewed as limiting the scope of the present invention. Reasonable variations, such as those occur to reasonable artisan, can be made herein without departing from the scope of the present invention.

### EXAMPLE 1

### Establishment of the GF-1 Cell Line

A vital sample of the immortal cell line derived from the fin tissue of grouper Epinephelus coioides, the GF-1 cell line, was deposited at the American Type Culture Collection (ATCC), 10801 University Blvd., Manassas, Va. 20110-2209, on Oct. 20, 1999, in compliance with the provisions of the Budapest Treaty for the International Recognition of the Deposit of Microorganisms for the Purpose of Patent Procedure. The assigned deposit number for this cell line is ATCC No. PTA-859. The viability of the GF-1 cell line was tested and confirmed on Nov. 1, 1999. The strain will be made available if a patent office signatory to the Budapest Treaty certifies one's right to receive, or if a U.S. Patent is issued citing the strain, and ATCC is instructed by the United States Patent & Trademark Office or the depositor to release the strain.

The GF-1 cell line was established and maintained as follows:

(1) Primary Culture

A grouper (Epinephelus coioides, Hamilton) weighing 1 kg was used for the establishment of the primary culture. The fish was dipped in 5% chlorex for 5 min, and then wiped with 70% alcohol. The fin was dissected from the body, and washed three times in a washing medium (containing L15 plus 400 IU/ml of penicillin, 400 μg/ml of streptomycin and 10 μg/ml of fungizone). After washing, the fin tissue was minced with scissors and then placed into 0.25% trypsin solution (0.25% trypsin and 0.2% EDTA in phosphate-buffered saline[PBS]). The tissue fragments in trypsin solution were slowly agitated with a magnetic stirrer at 4° C. At 30 min intervals, cells released from the tissue fragment were collected by centrifugation. Next, cells were re-suspended in a complete medium (containing L15 plus 20% of fetal bovine serum [FBS], 100 IU/ml of penicillin, 100 μg/ml of streptomycin, and 2.5 μg/ml of fungizone), transferred into a 25 cm² tissue culture flask and, finally, cultured at 28° C.

(2) Subculture and Maintenance

When the confluent monolayer of cells had formed in the primary culture, cells were dislodged from the flask surface by treating with 0.1% trypsin solution (containing 0.1% of trypsin and 0.2% of EDTA in PBS). The released cells were then transferred into two new flasks containing fresh L15 medium plus 20% of FBS. Cells were subcultured at a split ratio of 1:2. For the first ten subcultures of the GF-1 cells, a conditioned medium consisting of 50% old and 50% fresh medium was used. The concentration of FBS in the maintaining L15 medium was 10% for subcultures 11-70, and decreased to 5% after subcultures 70. Also, during the first twenty passages, GF-1 cells were subcultured at a 9-day interval. For the next 21^{th}-70^{th} passages, the GF-1 cells were subcultured at a 5-day interval. After 71 passages, GF-1 cells were subcultured at a 3-day interval.

(3) Test for Mycoplasma Contamination in the GF-1 Cell Line

The GF-1 cell line was propagated for three transfers in antibiotic-free L15-10% FBS and tested for the presence of bacteria, fungi, and mycoplasma. A mycoplasma stain kit (Flow Laboratories, U.S.A.) was used for mycoplasma testing.

(4) Test for the Viability of the GF-1 Cell Line

The viability of the GF-1 cell line was tested by first removing the cells from the flask. Then, the cells were separated from the medium by centrifugation, and re-suspended in a freezing medium consisting of 10% dimethyl sulfoxide (DMSO) and 90% FBS. Ampules (NUNC, Denmark) containing 5×10⁶ cells/ml/ampule were held at −20° C. for one hour, followed by staying at −70° C. overnight before being transferred to liquid nitrogen (−176° C.). After one month and one year, the ampules were thawed in a 30° C. water bath. The cells were separated from the freezing medium by centrifugation. The cells were re-suspended in L15-10% FBS. The viable cells were determined by trypan blue staining. The number of cells was counted using a hemacytometer. The thawed cells were re-seeded into a 25 cm² flask for further observation.

(5) Chromosome Number Distribution

The distribution of the chromosome numbers in GF-1 cells at subculture 50 and subculture 80 were studied using semi-confluent and actively growing cells. Cells were pre-treated with 0.1 μg/ml Colcemid (Gibco, Grand Island, N.Y.) for 5 hours at 28° C. before being dislodged with 0.1% of trypsin solution. After centrifugation at 1000 g for 10 min, the cells were re-suspended in a hypotonic solution (containing 8 parts of distilled water and 1 part of PBS) for 30 min. The cells were then partially fixed by adding several drops of Carnoy fixative (containing 1 part of Glacial acetic acid and 3 parts of 100% methanol). The partially fixed cells were further centrifuged at 800 g for 10 min at 4° C. The supernatant was discarded, and the cells were fixed in fresh, cold Carnoy fixative for 20 min. The suspension of fixed cells was dropped onto a 76×26 mm slide. The slide was air-dried and the cells were stained with 0.4% Giemsa stain (Sigma, St. Louis, Mo., USA) for 30 min. The chromosome numbers were observed and counted under an Olympus Vanox microscope.

(6) Plating Efficiency

The plating efficiency of the GF-1 cells was estimated at subcultures 50 and 80. Cells were seeded into a 25 cm² flask at a density of 100 cells per flask. Following 15 days of incubation, the medium was removed and the cell colonies were fixed with 70% ethanol and stained with 0.4% Giemsa. The colonies in each flask were then counted using an Olympus IM inverted microscope. Carp fin (CF), black porgy spleen (BPS-1), tilapia ovary (TO-2) and eel kidney (EK) cell lines were plated the same way as the GF-1 cell line for comparison purpose.

(7) Effects of FBS Concentration and Temperature on the Growth of the GF-1 Cells

The effects of the concentration of FBS on GF-1 cell growth were determined at subcultures 50 and 80. Two replicates were prepared for each FBS concentration. At selected intervals, two flasks were withdrawn from each concentration of FBS, and the mean number of cells was counted.

To determine the effects of temperature on the growth of GF-1 cells at subculture 80, replicated cell cultures in 25 cm² flasks containing L15-10% FBS were incubated at 18° C., 28° C. and 35° C. The mean number of the GF-1 cells from two replicated flasks at each temperature was counted at selected intervals.

Results:

Primary Culture and Subculture of the GF-1 Cells

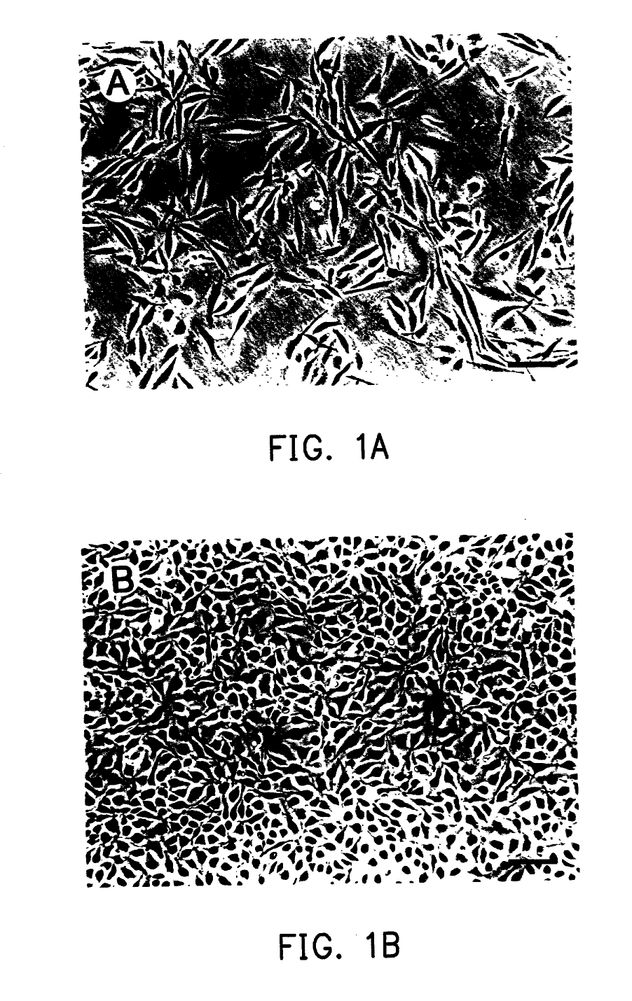

A monolayer of cells was formed in the primary culture approximately two weeks after the implantation. Fibroblast-like cells and epitheloid cells co-exist in the cell population (FIG. 1). The GF-1 cells have been successfully subcultured for more than 160 times since 1995, subsequently becoming a continuous cell line.

The GF-1 cells were subcultured at 9-day intervals in L15-20% of FBS during the first twenty subcultures, at 5-day intervals in L15-10% of FBS during the 21^{st}-70^{th} subcultures, and at 3-day intervals in L15-5% of FBS since subculture 71. Contact inhibition of the GF-1 cells was found in cultures before subculture 50, and gradually decreased between subculture 51 and 80.

The viability of the GF-1 cells at subculture 80 after one year and one month was 73%.

The re-seeded cells grew readily when incubated at 28° C. in L15-5% of FBS.

Chromosome Number

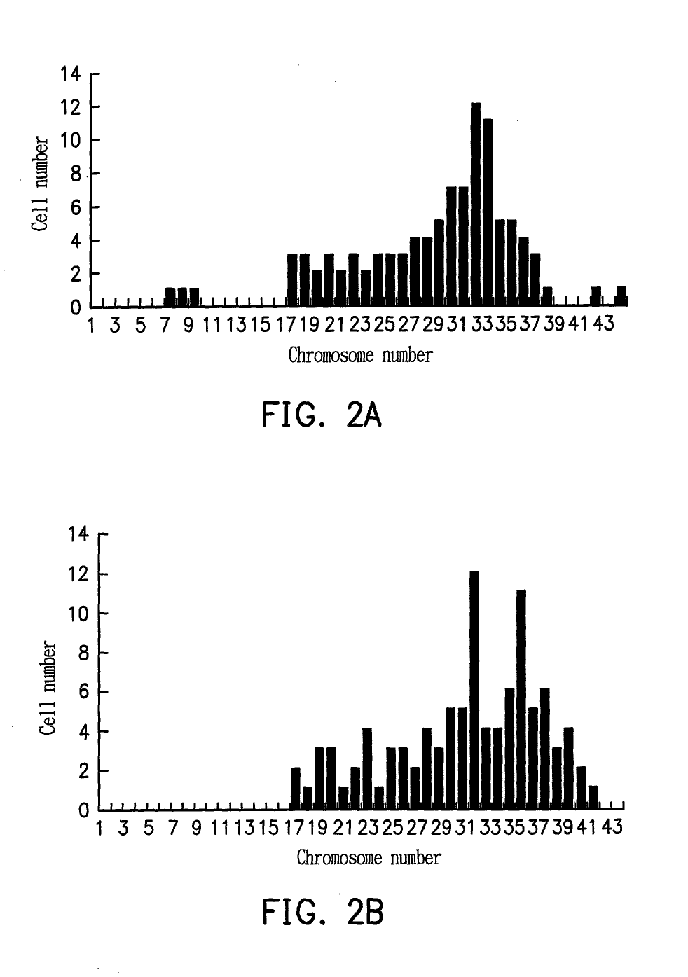

The chromosome number of the GF-1 cells at subculture 50 was distributed between 7 and 44 with the mode set at 32 (FIG. 2A). The chromosome number of the GF-1 cells at subculture 80 was distributed between 17 and 42 in 100 cells examined, and had a bimodal distribution with modes set at 32 and 36 (FIG. 2B). Both micro- and macro-chromosomes were found in metaphase-arrested cells.

Plating Efficiency

The plating efficiency of the GF-1 cells seeded at a density of 100 cells/flask was 21% at subculture 50 which increased to 80% at subculture 80. In comparison, the plating efficiencies of CF, BPS-1, TO-2 and EK cell lines seeded at a density of 100 cells/flask were 22%, 13%, 48%, and 63%, respectively. The increase in plating efficiency in GF-1 cells suggests the occurrence of transformation during subcultures 50-80.

Effects of FBS Concentration and Temperature on the Growth of the GF-1 Cells

FIG. 3 illustrates the effects of FBS concentration on the growth of the GF-1 cells at subcultures 50 and 80. The growth of the GF-1 cells at both subcultures 50 and 80 corresponded to the concentration of FBS, i.e., the higher the FBS concentration, the greater the growth of cells. However, when the growth rates of the GF-1 cells at subcultures 50 and 80 were compared, the GF-1 cells at subculture 80 demonstrated a much greater growth potential than those at subculture 50, especially when the FBS concentrations were at 2%, 5%, and 10%. For example, at day 4 of the cell cultures containing 10% of FBS, the GF-1 cells at subculture 50 have 3.5×10⁶ cells/25 cm² flask, whereas the GF-1 cells at subculture 80 have 5.0×10⁶ cells/25 cm² flask. These results suggest that the requirement of FBS for cell growth decreased at subculture 80, which is an indication that the transformation of cells had occurred during the period from subculture 50 to subculture 80.

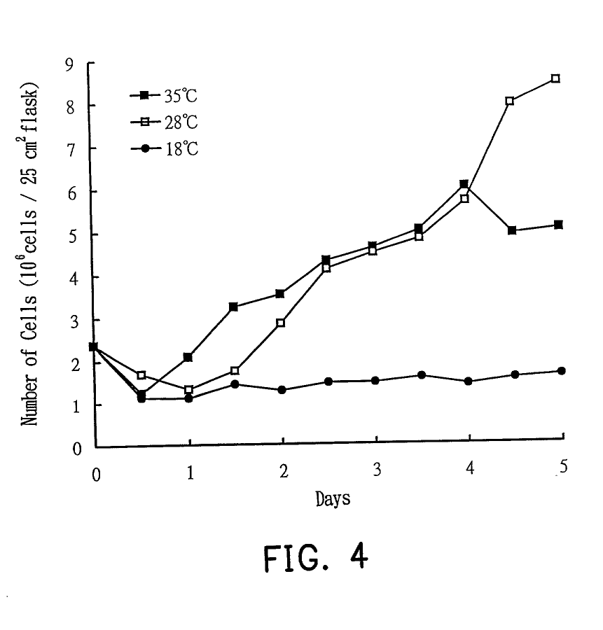

FIG. 4 illustrates the effect of temperature on the growth of the GF-1 cells at subculture 80. The results show that the GF-1 cells grew well at 28° C. and 35° C. However, the growth of the GF-1 cells cultured at 35° C. started to decline at day 4, suggesting that maintaining the cell culture at 35° C. may have long-term effects on cell growth. The GF-1 cells did not grow well at 18° C.

### EXAMPLE 2

### Methods for Producing Viruses Using the GF-1 Cell Line and Methods for Detecting the Viruses in the Cell Line

(1) Test for Susceptibility of the GF-1 Cells to Aquatic Viruses

Infectious pancreatic necrosis virus (IPNV, strain AB, SP, VR299 and EVE), hard clam reovirus (HCRV), eel herpes virus Formosa (EHVF) and nervous necrosis virus (NNV, GNNV isolate) were used to infect the GF-1 cells at subculture 80. The susceptibility to GNNV was also examined in BGF-1 cell line, which was derived from the fin of the banded grouper Epinephelus awoara.

Each of the monolayer GF-1 cells was inoculated with 0.5 ml of various aquatic virus with titer of 10³ TCID₅₀/0.1 ml. After a 30-min adsorption period, the cells from each flask were washed three times with PBS, followed by the addition of 5 ml of L15-2% FBS to each flask. The flasks were then incubated separately at 20° C. and 28° C. The supernatants of culture cells were collected and titrated for 6 days post viral infection.

(2) Multiplication and Purification of Aquatic Viruses in the GF-1 Cell Line

Viral isolate was inoculated at an MOI (multiplicity of infection) of 0.01 into the GF-1 cell line. When CPE appeared, the GF-1 cells were scraped into the medium and the cell debris was pelleted at 10000×g for 30 min (the first pellet). The supernatant was transferred to a bottle and polyethylene glycol (PEG, molecular weight 20000) and NaCl were added to reach a final concentration of 5% and 2.2% separately. The supernatant was then stirred for 4-6 hours at 4° C., and the virus particles were pelleted by centrifugation at 10000×g for 1 hour (the second pellet). The first pellet and the second pellet were re-suspended in a small amount of TNE buffer (0.1M Tris, 0.1M NaCl, 1 mM EDTA, pH 7.3), to which an equal volume of Freon 113 was added. The mixture was shaken vigorously for 5 min, and the emulsion was separated into the Freon and aqueous phase by centrifugation at 3000×g 10 min. The aqueous phase was collected, layered on a preformed 10-40% (w/w) CsCl gradient, and centrifuged at 160000×g for 20 hours. The visible virus band was collected, diluted with 10 ml of TNE buffer, and pelleted again by centrifugation at 150,000 g for 1 hours. The final pellet was resuspended in a small volume of TE buffer (0.1 M Tris, 1 mM EDTA, pH 7.3).

(3) Detection of Aguatic Viruses in the GF-1 Cell Line

In general, when a virus infects a cell line which is susceptible to the virus, a CPE of the cell culture can be observed within a couple of days after the infection. The appearance of CPE serves as evidence that the virus has successfully infected and multiplied in the cell line. The viral infection in the cell line can be further confirmed using an electron microscopic technique which is described as follows: The virus-infected cells were fixed in 2.5% glutaraldehyde in 0.1M of phosphate buffer at pH 7.4 and post-fixed in 1% of osmium tetraoxide. The cells were ultrathin sectioned. The ultrathin sections were stained with uranyl acetate-lead citrate and examined under a Hitachi H-600A electron microscope. The viral particles should appear as homogeneous, spherical particles in the cytoplasm of the cells.

There are also four methods that are directed to specific detection of NNV in the GF-1 cell line:

(A) Detection of NNV in the GF-1 Cells by Polymerase Chain Reaction (PCR) Amplification

A PCR amplification method was used to confirm that the GF-1 cells are able to proliferate NNV. The method required that the viral RNA be extracted from the supernatant of the NNV-infected cells after CPE appeared using a Rneasy™ mini kit (QIAGEN). For reverse transcription, extracted viral RNA was incubated at 42° C. for 30 min in 40 μl of 2.5×PCR buffer (25 mM of Tris-HCl, pH 8.8, 3.75 mM of MgCl₂, 125 mM of KCl, and 0.25% of Triton X-100) containing 2 U of MMLV reverse transcriptase (Promega), 0.4 U of RNsin (Promega), 0.25 mM of dNTP, and 0.5 μM of the reverse primer R3 (5′ CGAGTCAACACGGGTGAAGA 3′) (SEQ ID NO. 1). Following the cDNA synthesis, 40 μl of the cDNA mixture were diluted 2.5-fold with diethyl pyrocarbonate (DEPC)-treated H₂O (containing 0.025 U of DNA polymerase [Biometra], 0.1 mM of dNTP and 0.5 μM of the forward primer F2 [5′ CGTGTCAGTCATGTGTCGCT 3′] [SEQ ID NO.2]), and incubated in an automatic thermal cycler (TouchDown™ thermal cycler, Hybaid company). The target region for the primer set (F2, R3) is T4 (400 bp). The PCR products corresponding to T2 and T4 were amplified from the nucleic acids of NNV-infected GF-1 cells.

(B) Detection of NNV in the GF-1 Cells by Western Immunoblot

A western immunoblot method was used to specifically detect the NNV proteins. The viral sample was prepared as follows: NNV was inoculated into the GF-1 cells and incubated at 20-32° C. After 5 days of incubation, the NNV-infected cells were pelleted by centrifugation at 1000 g for 10 min. The cell pellets were loaded onto a 10% SDS-polyacrylamide gel. After electrophoresis, the proteins were blotted to an immobilon-P transfer membrane (Millipore), which was then soaked in a 3% skim milk tris buffered saline (TBS) for 1 hr. The membrane was then incubated with an antiserum against NNV for 1 hr at room temperature, washed with TBS, reacted with a peroxidase-conjugate goat system for 1 hr, and stained with a substrate containing 6 mg of 4-chloronaphthol in 20 ml of methanol and 60 μl of H₂O₂ in 100 ml of TBS.

(C) Detection of NNV in the GF-1 Cells by Enzyme-Linked Immunoabsorbent Assay (ELISA)

ELISA is an immunological method which uses an enzyme-labeled immunoreactant (antigen or antibody) and an immunosorbent (antigen or antibody bound to a solid) to identify specific serum or tissue antibodies or antigens. The ELISA test was conducted as follows: an effective amount of purified NNV proteins was coated onto a microtiter plate at 4° C. overnight. Then, 3% of bovine serum albumin (BSA) was added to the plate (used as blocking agent) and incubated at 37° C. for 1 hr. The plate was then washed 3 times with buffer. Next, a diluted rabbit anti-NNV serum was added to the plate and incubated at 37° C. for 1 hr. This was followed by the addition of goat anti-rabbit IgG-horseradish peroxidase serum at 37° C. for 1 hr and 3,3′,5,5′-tetramethyl benzidine was added for color development. The color reaction was stopped with 1 N H₂SO₄. The optical density of the wells in the microtiter plate was measured at 450 nm with an ELISA reader (Dynatech MR 5000).

(D) Detection of NNV in the GF-1 Cells by Immunofluorescent Staining

To detect the virus that proliferated in the GF-1 cells, cell cultures were fixed by 10% formalin for 12 hrs after viral infection. The fixed cell cultures were treated with 0.2% of Triton X-100 and washed with PBST (phosphate buffer with 0.05% Tween 20). The Triton-treated cell cultures were further washed with 3% of skim milk as blocking agent and then reacted with mouse anti-NNV serum. Finally, the antibody-treated cell cultures were stained with fluorescein isothiocyanate (FITC) conjugated goat anti-mouse antibodies.

Results:

Table 1 summarizes the results of virus susceptibilities of the GF-1 cells to IPNV (AB, SP, VR299, EVE strains), HCRV, EHVF and NNV (GNNV isolate), which were determined by first observing the appearance of CPE in the cells after the viral inoculation, followed by the determination of viral titers (TCID₅₀/ml).

TABLE 1
Viral Susceptibilities of GF-1 Cells at Subculture 80

| | | Initial Viral | | | Virus Yield/ml | |
|---|---|---|---|---|---|---|
| | | Inoculum | CPE | | (TCID₅₀/ml) | |
| Cell line | Virus | (TCID₅₀) | 28° C. | 20° C. | 28° C. | 20° C. |
| GF | IPNV | | | | | |
| AB | 10³ | — | + | ND | 10^{9.5} | |
| SP | 10³ | — | + | ND | 10^{10.8} | |
| VR299 | 10³ | — | + | ND | 10^{9.8} | |
| EVE | 10³ | — | + | ND | 10^{9.6} | |
| HCRV | 10³ | — | + | ND | 10^{11.0} | |
| EHVF | 10³ | + | + | 10^{8.1} | 10^{7.0} | |
| GNNV | 10³ | + | — | 10^{8.3} | ND | |

ND: Not done.
+: Cytopathic effect (CPE) was observed.

As shown in Table 1, for the IPNV strains and HCRV, CPE appear only when the cells are incubated at 20° C. For EHVF, CPE appears at both 20° C. and 28° C. However, for GNNV, CPE appears at 28° C. The yields of the viruses in GF-1 cells at subculture 80, which are ranged between 10^{7.0} 73787 (as for EHVF at 20° C.) and 10^{11.0} (as for HCRV at 20° C.), are extremely high.

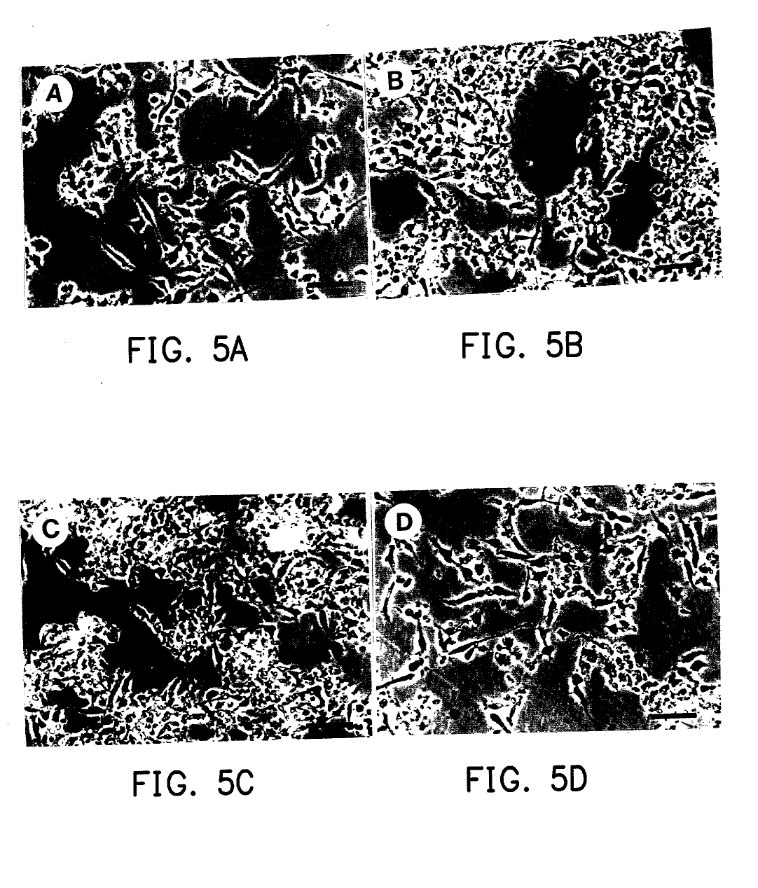

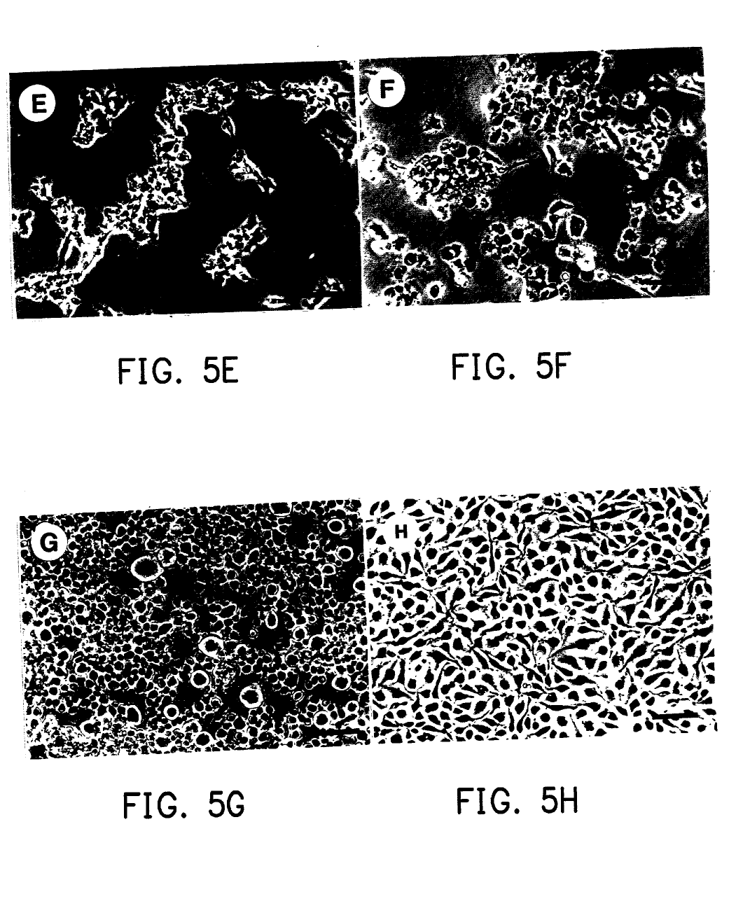

Typically, for an aquatic virus such as GNNV, CPE began at the 3rd day of infection when some rounded, granular, refractile cells began to appear in the cell culture (FIG. 5). Soon more and more cells became round and swollen. The swollen cells became larger and finally started to detach from the cell culture and float in the culture media. Most of the detached cells were completely disintegrated. The culture fluid from cell culture showing CPE could transmit other GF-1 cells. This experiment also tested the susceptibility of BGF-1 cell line (derived from the fin of the banded grouper Epinephelus awoara) to GNNV. The results showed that no CPE was found after the viral infection.

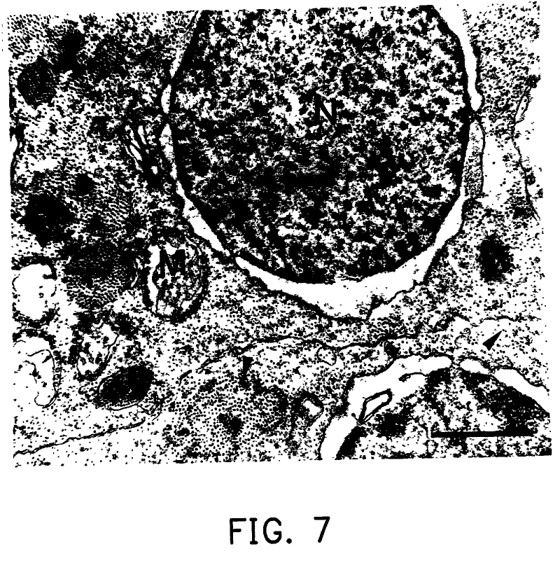

Typically, for an aquatic virus such as GNNV, the virus could be observed in the cytoplasm of the GF-1 cells under electron microscope as numerous non-enveloped, homogeneous, spherical to icosahedral particles with diameter of 20-25 nm (FIG. 7). Some of the viral particles were included in the inclusion bodies and the others could be found in the cytoplasm (FIG. 7). The isolated viral particles could be further purified by CsCl density gradient centrifugation. Using GNNV as an example, the purified virus was a non-enveloped icosahedral virion particle with the diameter of 20-25 nm. The buoyant density of GNNV in CsCl was 1.34 g/cm³.

As for IPNV, the virus can be concentrated by polyethylene glycol-NaCl precipitation and purified by Freon extraction and isopycnic CsCl gradient centrifugation, as described by Bootland et al., J. Fish Diseases (1995) 18:449-458. The buoyant density of IPNV in CsCl is 1.33 g/cm³.

In addition to the findings of CPE in the GF-1 cells, the existence of an aquatic virus in the GF-1 cells and the capability of the cells to multiply the virus can be further confirmed by four methods: (1) the PCR method; (2) the Western immunoblot method; (3) the ELISA method; and (4) the immunofluorescent staining method.

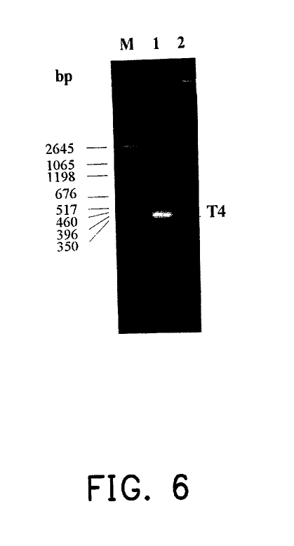

Using GNNV as an example, the PCR method could be accomplished by choosing a pair of primers, i.e., R3 (SEQ ID NO.1) and F2 (SEQ ID NO.2), for PCR amplification. The target fragment T4 exists in fish nodavirus. Therefore, the PCR method using F2 and R3 was specific to fish nodavirus, not just GNNV. The results of the PCR study showed that GNNV could be replicated in the GF-1 cells and released into the supernatant of culture cells (FIG. 6).

The Western immunoblot using mouse anti-GNNV serum demonstrated that viral proteins were present in the GNNV-infected cells cultured at 20-32° C., suggesting that the viral MRNA could be successfully translated into viral polypeptides within the host cells when the culture was maintained at 20-32° C.

The ELISA and immunofluorescent staining methods also showed positive reactions with the anti-GNNV serum, indicating that GNNV could be multiplied in the GF-1 cells.

### EXAMPLE 3

### Production of Anti-NNV Antibodies

(1) Production of Anti-NNV Antibodies

Polyclonal antibodies can be produced in accordance with conventional methods, e.g., by sequential injections of the purified NNV immunogen into a suitable animal such as a rabbit, rat, or mouse. For example, a suitable amount of the NNV immunogen can be injected intravenously, subcutaneously, or intraperitoneally to a rabbit and boosted twice or more at 2 or 3 week intervals. The injection may contain a suitable amount of Freund's complete or incomplete adjuvant, if necessary.

For the production of monoclonal antibodies, immunizing mice is preferred. Three or four days after the final boost, spleen cells of mice can be separated and fused with myeloma cells, e.g., SP2/0-Ag14 myeloma cells (ATCC CRL 1581), in accordance with a conventional method described by Mishell and Shiigi (Selected Mthods in Cellular Immunology, W. H. Freeman & Company, 1980). The spleen cells and the myeloma cells can be used in a ratio ranging from 1:1 to 1:4. A fusion-promoting agent, e.g., polyethylene glycol (PEG) 4000, may be employed for accelerating the cell fusion. A medium suitable for use in the cell fusion step may be RPMI 1640 (Gibco BRL, Life Technologies, Inc.) and the medium generally contains 10-15% (v/v) fetal bovine serum (FBS).

The fused cells can be cultured in the RPMI1640-15% FBS, supplemented with hypoxanthine, thymidine and aminopterin, and after seven to ten days, positive hybridoma clones producing antibodies specific for NNV can be selected by ELISA assay using the culture supernatant. Further selection of positive clones can be accomplished by using conventional methods, e.g., the limiting dilution technique, the plaque method, spot method, agglutination assay and autoradiographic immunoassay.

(2) Purification of Antibodies

Antibody can be purified by conventional immunoglobulin purification procedures such as ammonium sulfate precipitation, gel electrophoresis, dialysis, affinity chromatography, and ultrafiltration. Ion exchange, size exclusion hydroxylapatite, or hydrophobic interaction chromatography can be employed, either alone or in combination. Light and heavy chain can be carried out using gel electrophoretic techniques or isoelectric focusing, as well as other techniques known in the art.

### EXAMPLE 4

### Production of Vaccines

Preparation of NNV or IPNV Vaccines Using Inactivated NNV or IPNV

The vaccines of the present invention can be administered as inactivated virus vaccines, which encompass any methods now known or hereafter developed for killing or inactivation.

Complete virus inactivation can be determined by the absence of cytopathic effect (CPE) in GF-1 cells. The protein content of the inactivated virus can be measured by Lowry assay. There are many ways to prepare inactivated viruses fore use in vaccine production. The most favorable methods for inactivating viruses are by heat treatment or formalin treatment. For example, a 60-100° C. treatment of NNV for at least 1 hour can totally block the NNV infectivity. Also, a formalin treatment of IPNV by incubating IPNV for 7 days at 22° C. in a final concentration of about 0.5% formalin (pH 7.4), followed by dialysis against distilled water for 48 hours, can inactivate IPNV. The infectivity of NNV can also be blocked by treating NNV with extreme pH not higher than 3.0 or not less than 10.0).

After the inactivated virus is prepared, for intraperitoneal or intramuscular vaccination, the inactivated NNV can be emulsified in Freund's incomplete adjuvant (FIA) using a mixer for several minutes. The vaccine can then be injected into the fish (the primary injection). Booster injections can be given to the fish 30-45 days after the primary injection. Normally, the booster injection consists of about one half of the volume of the vaccine used in the primary injection. The fish then can receive a secondary boost 10 days after the first booster shot is administered. The serum samples from the fish at various time points can be taken for titer determination.

For orally administered vaccine, an enteric coating containing non-toxic polymeric materials can be added to the vaccine. The preferable enteric coating materials are the ones that can resist dissolution at the pH of the stomach but can be dissolved once the material passes from the stomach to the pyloric caecum and intestines. For example, cellulose acetate phthalate, hydroxypropylmethyl cellulose phthalate, carboxymethylethyl cellulose, hydroxypropylmethyl cellulose acetate succinate, cellulose acetate trimellitate, polyvinyl acetate phthalate, EUDAGRIT L-30D and 1100-55, EUDAGRIT L 12.5 and L 100, EUDRAGIT E, RL, RS and NE are among the preferred materials. Additional materials can be used in combination with the enteric coating materials. For instance, plasticizers (such as polyethylene glycol 200, 400, 1000, 4000, 6000, propylene glycol, PVPK-90, glycerin or glycerol, diethyl phthalate, oleic acid, isopropyl myristate, liquid paraffin or mineral oil, triacetin, glycerol monostearate, dibutyl sebacate, triethyl citrate, tributyl citrate, acetylated monoglyceride, dibutyl phthalate, acetyl tributyl citrate, castor oil, and glycerol tributyrate); disintegrants (such as sodium starch glycolate); adjuvants (such as immunostimulants [e.g., beta glucan]); binders (such as starch, polyvinyl pyrrolidone, polyvinyl alcohol); diluents (such as lactose); lubricants (such as magnesium stearate) etc. can all be used with the enteric coatings. For oral administration, fish can receive the vaccine on an every-other-day basis for a total of thirty days. The effects of the vaccines can be monitored by the use of ELISA.

Administering vaccines orally to fish is superior to injecting vaccines to fish, because the oral administration method is not contrained by the size of the fish that can be handled, and can reduce the stress on the fish associated with immersion and intraperitoneal injection. Furthermore, oral vaccines offer the additional advantages of stimulating the gut-associated lymphoid tissue to a greater extent than does intraperitoneal injection.

To collect the virus for inactivation preparation, the virus was first mass-produced in GF-1 cell line according to EXAMPLE 2. The virus was then separated and purified by CsCl density gradient centrifugation as described in EXAMPLE 2 (2). Alternatively, the virus can be collected by low speed centrifugation to remove the cell debris.

The present invention has been described with reference to several preferred embodiments. Other embodiments of the invention will be apparent to those skilled in the art from the consideration of this specification or practice of the invention disclosed herein. It is intended that the specification and examples contained herein be considered as exemplary only, with the true scope and spirit of the invention being indicated by the following claims:

What is claimed is:

1. A vaccine for immunizing susceptible fish against viral infection comprising:
an immunogenically effective amount of a virus; wherein said virus is produced in an immortal cell line from Epinephelus cofoides having an ATCC deposit No. PTA-859; and wherein said virus is modified to become non-infective to said susceptible fish.

2. The vaccine according to claim 1, wherein said virus is a nervous necrosis virus (NNV).

3. The vaccine according to claim 2, wherein said susceptible fish is one selected from the group consisting of parrotfish, sea bass, turbot, grouper, stripped jack, tiger puffer, berfin flounder, halibut, barramundi, and spotted wolffish.

4. The vaccine according to claim 1, wherein said virus is an infectious pancreatic necrosis virus (IPNV).

5. The vaccine according to claim 4, wherein said susceptible fish is one selected from the group consisting of trout, salmon, carp, perch, pike, and eel.

6. The vaccine according to claim 1, wherein said virus is a inactivated virus.

7. The vaccine according to claim 6, wherein said vaccine is orally administered to or intraperitoneally or intramuscularly injected into said susceptible fish.

8. A method for immunizing susceptible fish against viral infection comprising:
administering to said susceptible fish a vaccine comprising a non-infective virus; wherein said virus is produced in the immortal cell line according to claim 1.

9. The method according to claim 8, wherein said virus is a nervous necrosis virus (NNV).

10. The method according to claim 9, wherein said susceptible fish is one selected from the group consisting of parrotfish, sea bass, turbot, grouper, stripped jack, tiger puffer, berfin flounder, halibut, barramundi, and spotted wolffish.

11. The method according to claim 8, wherein said virus is an infectious pancreatic necrosis virus (IPNV).

12. The method according to claim 11, wherein said susceptible fish is one selected from the group consisting of trout, salmon, carp, perch, pike, and eel.

13. The method according to claim 11, wherein said non-infective virus is an inactivated virus.

14. The method according to claim 13, wherein said vaccine is orally administered to or intraperitoneally or intramuscularly injected into said susceptible fish
